(19) United States
(12) Patent Application Publication
(10) Pub. No.: US 2015/0366252 A1
(43) Pub. Date: Dec. 24, 2015

(54) SWEETENER ENHANCERS AND METHODS FOR USING THE SAME

(71) Applicant: International Flavors & Fragrances Inc., New York, NY (US)

(72) Inventors: Jianke Li, Highland Park, NJ (US); Tingwei Cai, Holmdel, NJ (US); Neelima Mannava, Manalapan, NJ (US); Jung-A Kim, Edgewater, NJ (US); Xiaodong Li, Middletown, NJ (US); Xiaoqing Tang, Holmdel, NJ (US)

(73) Assignee: INTERNATIONAL FLAVORS & FRAGRANCES INC., New York, NY (US)

(21) Appl. No.: 14/692,859

(22) Filed: Apr. 22, 2015

Related U.S. Application Data
(60) Provisional application No. 62/016,245, filed on Jun. 24, 2014.

(51) Int. Cl. A23L 1/22; A61K 8/49; A23L 1/236; A61K 47/22; A23G 4/10; C07D 417/12; A61Q 11/00

(52) U.S. Cl. CPC A23L 1/22091; C07D 417/12; A61K 8/4926; A61Q 11/00; A61K 47/22; A23G 4/10; A23L 1/2363; A23V 2002/00

(57) ABSTRACT

A compound of Formula I:

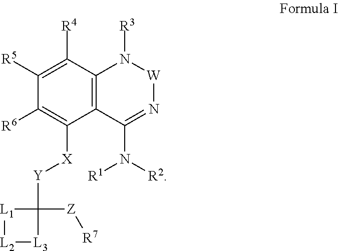

In this formula, each of R¹, R², R³, R⁴, R⁵, R⁶, R⁷, L¹, L², L³, W, X, Y, and Z is defined herein. Also disclosed are a composition containing a compound of Formula I and a method for enhancing the sweet taste of a consumable product using such a compound.

### CROSS-REFERENCE TO RELATED APPLICATIONS

This application claims priority to U.S. provisional application, Ser. No. 62/016,245 filed on Jun. 24, 2014, the content of which is incorporated herein by reference in entirety.

### BACKGROUND

Sugars are significant sources of extra calories in diet and have been linked to obesity, diabetes, cardiovascular disease, dementia, macular degeneration, and tooth decay.

High-intensity sweeteners have been used to reduce the use of sugars. Examples of these sweeteners include compounds such as sucralose, aspartame, and saccharin. However, the sensation of sweetness (i.e., sweetness profile) caused by these compounds is notably different from natural sugars including glucose and sucrose. More specifically, sucralose and aspartame have delayed onset and lingering sweetness and saccharin has a bitter aftertaste. Using these sweeteners cannot achieve the same mouthfeel as natural sugars like glucose and sucrose.

There is a need to develop compositions having sweetness profile very close to that of natural sugars.

### SUMMARY

The present invention is based on the discovery that compounds of Formula I below can significantly enhance the sweet taste of natural sugars, e.g., glucose.

One aspect of this invention relates to compounds of Formula I:

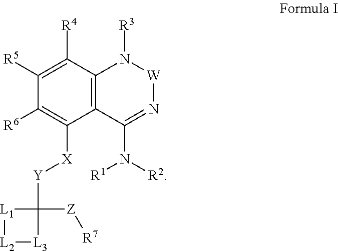

In this formula, W is S(O)₂ or C(O); X is O, S, or NR^{a}, R^{a} being H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto; Y is C₁-C₃ alkylene; Z is CONR^{b}, R^{b} being H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto; each of L¹, L², and L³, independently, is a bond, O, S, CO, C₁-C₆ alkylene, C₂-C₆ alkenylene, CONR, R^{d}NCONR^{e}, R^{f}NC(═NR^{g})NR^{h}, CRⁱR^{j}, or NR^{k}, provided that L¹, L², L³, and the carbon atom to which L¹ and L³ are attached form a 3- to 8-membered (e.g., 5- or 6-membered) cycloalkyl or heterocycloalkyl, each of R^{e}, R^{d}, R^{e}, R^{f}, R^{g}, R^{h}, and R^{k}, independently, being H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto; and each of Rⁱ and R^{j}, independently, being H, OH, SH, CN, NO₂, NH₂, halo, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto; each of R¹ and R², independently, is H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₃-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto; or R¹, R², and the N atom they attached to, together, form a 3- to 8-membered (e.g., 5- or 6-membered) heterocycloalkyl or heteroaryl ring; each of R³ and R⁷, independently, is H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₃-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, aryl-sulfonamide, heteroarylsulfonamide, C₁-C₁₀ alkylmercapto, or arylmercapto; and each of R⁴, R⁵, and R⁶, independently, is H, OH, SH, CN, NO₂, NH₂, halo, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto.

In these compound, each of cycloalkyl, heterocycloalkyl, aryl, arylalkyl, heteroaryl, and heteroarylalkyl, independently, is unsubstituted or substituted with C₁-C₁₀ alkyl, C₂-C₁₀ alkenyl, C₂-C₁₀ alkynyl, C₃-C₈ cycloalkyl, C₁-C₈ heterocycloalkyl, C₁-C₁₀ alkoxy, C₁-C₁₀ alkylthio, aryl, aryloxy, arylthio, heteroaryl, heteroaryloxy, heteroarylthio, amino, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, hydroxyl, halogen, mercapto, C₁-C₁₀ alkylmercapto, arylmercapto, cyano, nitro, acyl, acyloxy, carboxyl, amido, carbamoyl, or carboxylic ester; and each of alkyl, alkenyl, alkynyl, alkylene, and alkenylene is unsubstituted or substituted with C₃-C₈ cycloalkyl, C₁-C₈ heterocycloalkyl, C₁-C₁₀ alkoxy, C₁-C₁₀ alkylthio, aryl, aryloxy, arylthio, heteroaryl, heteroaryloxy, heteroarylthio, amino, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, hydroxyl, halogen, mercapto, C₁-C₁₀ alkylmercapto, arylmercapto, cyano, nitro, acyl, acyloxy, carboxyl, amido, carbamoyl, or carboxylic ester.

In one subset of these compounds, each of R¹, R², R³, R⁴, R⁵, and R⁶ is H.

In any compounds described above, Y can be CH₂, W can be SO₂, X can be 0, and L¹, L², L³, and the atom to which L¹ and L³ attached form a 5 or 6-membered cycloalkyl or heterocycloalkyl ring. The above-described compounds may have one or more of the following features: L¹ is C(O)NH, Z is C(O)NH, and R⁷ is C₃-C₁₀ cycloalkyl, C₁-C₁₀ heterocycloalkyl, C₁-C₁₀ alkyl, or C₁-C₁₀ heteroalkyl.

“Acyl” means a —C(O)R^{a1} radical where R^{a1} is alkyl, cyanoalkyl, haloalkyl, alkoxyalkyl, cycloalkyl, cycloalkyl, aryl, aralkyl, heteroaryl, heteroaralkyl, optionally substituted heterocycloalkyl, or optionally substituted heterocycloalkylalkyl, as defined herein, e.g., acetyl, benzoyl, and the like. “Acyloxy” means —OC(O)R^{a2} wherein R^{a2} is as defined immediately above.

“Alkyl” means a linear saturated monovalent hydrocarbon radical of one to twenty (e.g., 1-10 and 1-6) carbon atoms or a branched saturated monovalent hydrocarbon radical of three to twenty (e.g., 3-10 and 3-6) carbon atoms, e.g., methyl, ethyl, propyl, 2-propyl, butyl (including all isomeric forms), pentyl (including all isomeric forms), hexyl (including all isomeric forms), and the like. The term “alkylene” refers to bivalent alkyl. Examples include —CH₂—, —CH₂CH₂—, —CH₂CH₂CH₂—, —CH₂(CH₃)CH₂—, and —CH₂CH₂CH₂CH₂—.

The term “alkenyl” refers to a linear or branched hydrocarbon radical of one to twenty (e.g., 2-10 and 2-6) carbon atoms and one or more double bonds. Examples include —CH═CH₂, —CH═CHCH₃, —CH₂CH═CH₂, and —CH₂CH═CHCH₃. The term “alkenylene” refers to bivalent alkenyl. The term “alkynyl” refers to a linear or branched hydrocarbon radical of one to twenty (e.g., 2-10 and 2-6) carbon atoms and one or more triple bonds. The term “alkynylene” refers to bivalent alkynyl.

“Alkoxy” means a radical —OR^{a3} where R^{a3} is alkyl, alkenyl, or alkynyl as defined herein. Examples include methoxy, ethoxy, propoxy, 2-propoxy, n-, iso-, or tert-butoxy, and the like. “Alkylthio” means an —SR^{a4} radical where R^{a4} is alkyl, alkenyl, or alkynyl as defined herein, e.g., methylthio, ethylthio, propylthio, butylthio, and the like. “Amino” means an —NH₂ radical or an N-oxide derivative, or a protected derivative thereof such as —NH→O, —NHBoc, or —NHCbz, and the like. Boc refers to tert-butoxycarbonyl and Cbz refers to carboxybenzyl.

“Aryl” means a monovalent, monocyclic, fused bicyclic, or tricyclic hydrocarbon radical of 6 to 14 ring atoms, wherein the ring comprising a monocyclic radical ring is aromatic and wherein at least one of the fused rings containing a bicyclic or tricyclic radical is aromatic. Unless otherwise stated, the valency of the group may be located on any atom of any ring within the radical, valency rules permitting. More specifically, the term aryl includes, but is not limited to, phenyl, naphthyl, anthracenyl, indanyl (including, for example, indan-5-yl, or indan-2-yl, and the like), tetrahydronaphthyl (including, for example, tetrahydronaphth-1-yl, tetrahydronaphth-2-yl, and the like), and the like. Unless indicated otherwise, aryl is unsubstituted or may be substituted with one or more “ring system substituents” which may be the same or different, and are as defined herein.

“Aryloxy” means —OR^{a5} where R^{a5} is aryl as defined herein. Examples include phenoxy and naphthoxy. Arylthio means —SR^{a6} where R^{a6} is aryl as defined herein. “Arylalkyl” means an alkyl radical substituted with aryl, as defined herein. Examples include benzyl. “Carboxyl” means a —C(O)OH radical.

“Cycloalkyl” means a monocyclic, fused bicyclic, or fused tricyclic, saturated or unsaturated, monovalent hydrocarbon radical of three to fourteen carbon ring atoms. Unless otherwise stated, the valency of the group may be located on any atom of any ring within the radical, valency rules permitting. More specifically, the term cycloalkyl includes, but is not limited to, cyclopropyl, cyclobutyl, cyclopentyl, cyclohexyl, cycloheptyl, decahydronaphthyl (e.g., decahydronaphth-1-yl, decahydronaphth-2-yl, and the like), norbornyl, adamantly, cyclopropenyl, cyclobutenyl, cyclopentenyl, cyclohexenyl, and the like. The cycloalkyl ring is unsubstituted or may be substituted with one or more “ring system substituents” which may be the same or different, and are as defined herein.

“Halogen” or “halo” means fluorine, chlorine, bromine, or iodine.

“Heteroalkyl” means an alkyl radical as defined herein where one or more carbon atoms are replaced by an oxygen, nitrogen, phosphorous, or sulphur atom. Examples include an alkoxy group (e.g., methoxy, ethoxy, propoxy, iso-propoxy, butoxy, and tert-butoxy), an alkoxyalkyl group (e.g., methoxymethyl, ethoxymethyl, 1-methoxyethyl, 1-ethoxyethyl, 2-methoxyethyl, and 2-ethoxyethyl), an alkylamino group (e.g., methylamino, ethylamino, propylamino, isopropylamino, dimethylamino, and diethylamino), an alkylthio group (e.g., methylthio, ethylthio, and isopropylthio), and a cyano group. Any heteroalkyl group as defined herein may be substituted with one, two or more substituents, for example, F, Cl, Br, I, NH₂, OH, SH, NO₂, cyclohexyl, 2-piperidonyl, 3-piperidonyl, and 4-piperidonyl.

“Heterocycloalkyl” means a saturated or unsaturated, nonaromatic, 5-8 membered monocyclic, 8-12 membered bicyclic, or 11-14 membered tricyclic ring system having one or more heteroatoms (e.g., O, N, P, and S). Examples include 3-piperidonyl, 4-piperidonyl, 5-piperidinonyl, 6-piperidinonyl, piperidinyl, piperazinyl, imidazolidinyl, imidazolidonyl, azepanyl, pyrrolidinyl, 2-pyrrolidonyl, 3-pyrrolidonyl, dihydrothiadiazolyl, dioxanyl, morpholinyl, 2-morpholinonyl, 3-morpholinonyl, tetra-hydropuranyl, and tetrahydrofuranyl. The term “heterocyclo-alkylene” refers to bivalent heterocycloalkyl. The term “heterocycloalkenyl” refers to a nonaromatic 5-8 membered monocyclic, 8-12 membered bicyclic, or 11-14 membered tricyclic ring system having one or more heteroatoms (e.g., O, N, P, and S) and one or more double bond.

“Heteroaryl” means a monocyclic or fused bicyclic, monovalent radical of 5 to 12 ring atoms containing one or more, preferably one, two, three, or four ring heteroatoms independently selected from the group of N, O, P(O)ₘ, —Si (where Si is substituted with alkyl and one additional group selected from alkyl, alkenyl, cycloalkyl-alkyl, aryl, aralkyl, heteroaralkyl, and optionally substituted heterocycloalkylalkyl), and S(O)ₙ, where m is 1 or 2 and n is 0, 1, or 2, the remaining ring atoms being carbon, wherein the ring comprising a monocyclic radical is aromatic and wherein at least one of the fused rings comprising the bicyclic radical is aromatic. One or two ring carbon atoms can optionally be replaced by a —C(O)—, —C(S)—, or C(═NH)— group. Unless otherwise stated, the valency may be located on any atom of any ring of the heteroaryl group, valency rules permitting. More specifically, the term heteroaryl includes, but is not limited to, phthalimidyl, pyridinyl, pyrrolyl, pyrazolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), pyrazinyl, pyrimidinyl, pyridazinyl, oxazolyl, isoxazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, methylenedioxyphenyl (including, for example, methylenedioxyphen-5-yl), and the derivatives thereof, or N-oxide or a protected derivative thereof. The heteroaryl ring is unsubstituted or may be substituted with one, two, or three “ring system substituents” which may be the same or different, and are as defined herein.

“Heteroaryloxy” means —OR^{a7} wherein R^{a7} is heteroaryl as defined herein. “Heteroarylthio” means —SR^{a8} wherein R^{a8} is heteroaryl as defined herein. “Heteroarylalkyl” means an alkyl radical, as defined herein, substituted with at least one, preferably one or two, heteroaryl group(s) as defined herein, e.g., pyridinylmethyl, furanylmethyl, or chloropyridinylmethyl, and the like.

“Ring system substituent” means a substituent attached to an aromatic or non-aromatic ring system, which, for example, replaces an available hydrogen on the ring system. Ring system substituents may be the same or different, each being independently being, e.g., —C(═NH)(NH₂), —NHC(═NH)(NH₂), alkyl, alkenyl, alkynyl, alkoxy, acyl, alkylcarbonylamino, carboxy, carboxyalkyl, alkoxycarbonyl, alkoxycarbonylalkyl, cyano, nitro, alkylthio, halo, haloalkyl, haloalkoxy, amino, alkylamino, dialkylamino, aminocarbonyl, alkyl aminocarbonyl, dialkylaminocarbonyl, alkyl sulfonyl, cycloalkylsulfonyl, alkylsulfonylamino, alkylaminosulfonyl, haloalkylamino, oxo, hydroxy, hydroxyalkyl, hydroxyalkyloxy, hydroxyalkyloxyalkyl, alkoxyalkyloxyalkyl, aryl, heteroaryl, cycloalkyl, cycloalkylamino, cycloalkyloxy, heteroaralkyloxy, aminoalkyl, aminoalkyloxy, alkoxyalkyl, alkoxyalkylcarbonyl, alkoxyalkyloxy, haloalkoxyalkyl, aryloxyalkyl, heteroaryloxyalkyl, heterocycloalkyl, heterocycloalkyloxyalkyl, heterocycloalkylalkyl, heterocycloalkylalkyloxy, or heterocycloalkyloxy. “Ring system substituent” may also mean a single moiety that simultaneously replaces two available hydrogens on two adjacent carbon atoms (one H on each carbon) on a ring system. Examples of such moieties are methylenedioxy and ethylenedioxy.

The compounds described above also include their salts and solvates, if applicable. A salt can be formed between an anion and a positively charged group (e.g., amino) on a compound; examples of a suitable anion include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. A salt can also be formed between a cation and a negatively charged group; examples of a suitable cation include sodium ion, potassium ion, magnesium ion, calcium ion, and an ammonium cation such as tetramethylammonium ion. A salt further includes those containing quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine.

Another aspect of this invention relates to a composition comprising a sweetener and any one of the compounds described above. These compounds are capable of enhancing the sweet taste of the sweetener, which can be glucose. The content of the compound in the composition can be 1 part per billion or greater by weight (e.g., 100 parts per billion (“ppb”) to 100 parts per million (“ppm”), and 300 ppb to 50 ppm). The composition of this invention can further contain a material selected from the group consisting of foodstuff, a chewing gum, a dental or oral hygiene product, and a medicinal product.

A further aspect of this invention related to a method for enhancing the sweet taste of a consumable product. The method includes mixing the consumable product with any of the compounds described above. The consumable product contains a sweetener and this compound is capable of enhancing the sweet taste of the sweetener.

An additional aspect of this invention relates to use of any one of the compounds described above for enhancing the sweet taste of a sweetener (e.g., glucose).

The details of one or more embodiments of the invention are set forth in the description below. Other features, objects, and advantages of the invention will be apparent from the description and the claims.

### DETAILED DESCRIPTION OF THE INVENTION

The present invention is directed to a chemosensory receptor ligand enhancer, which has been shown to significant increase the sweet taste of sweeteners including glucose.

Shown below are three exemplary compounds of this invention:

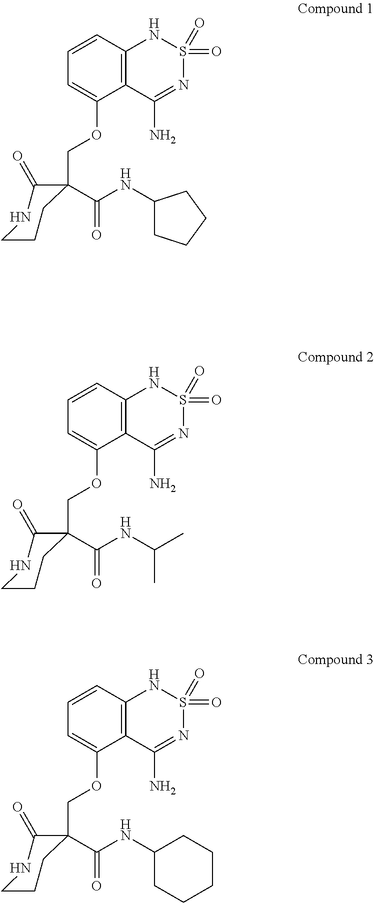 [SMILES: NC1=NS(=O)(=O)Nc2cccc(OCC3(C(=O)NC4CCCC4)CCCNC3=O)c21 ; CC(C)NC(=O)C1(COc2cccc3c2C(N)=NS(=O)(=O)N3)CCCNC1=O ; NC1=NS(=O)(=O)Nc2cccc(OCC3(C(=O)NC4CCCCC4)CCCNC3=O)c21]

The route shown in Scheme I below exemplifies synthesis of certain compounds of this invention.

Following known procedures, a reaction between a compound of Formula A and a compound of Formula B gives a compound of Formula C. See Meshram et. al., Synth. Commun. 2010, 40, 2122; Woiwode et al., J. Org. Chem. 1998, 63, 9594; Klaubert et al., J. Med. Chem. 1981, 24, 742; and Chan et al., J. Med. Chem. 2001, 44, 1866. Treating the compound of Formula C with R³NH₂ leads to a compound of Formula D using the procedures described in Joensson et al., J. Med. Chem. 2004, 47, 2075 and Bruncko et al., J. Med. Chem. 2007, 50, 641. The nitrile group in the compound of Formula D is converted to an amidine group to yield a compound of Formula E. Examples of conversion reactions are described in Anbazhagan et al., Synthesis 2003, 2467; Garigipati, Tetr. Lett. 1990, 31, 1969; Schaefer et al., J. Org. Chem. 1962, 27, 1255; Packiarajan et al., Bioorg. Med. Chem. Lett. 2012, 22, 5658; Medwid et al., J. Med. Chem. 1990, 33, 1230; Lorz et al., J. Am. Chem. Soc. 1948, 70, 1904; Vitale et al., Tetr. 2011, 67, 6944; and Schnur, J. Org. Chem. 1979, 44, 3726. The compound of Formula E is then cyclized with triphosgene (see Cortez et al., Synth. Commun. 1991, 21, 285 and Apfel et al., J. Med. Chem. 2001, 44, 1847), or with CO(OEt)₂ (See Han et al., Org. Lett. 2007, 9, 1517 and Paz et al., J. Org. Chem. 2010, 75, 3037.), or with 1,1′-carbonyldiimidazole (See Pascal et al., Eur. J. Org. Chem. 2000, 22, 3755 and Rzasa et al., Bioorg. Med. Chem. 2007, 15, 6574.), or with H₂NCONH₂ (See Mayer et al., J. Med. Chem. 2000, 43, 3653), or with H₂NSO₂NH₂ (See Kim et al., Bioorg. Med. Chem. Lett. 2008, 18, 5815; Hannam et al., Synlett 2006, 833; Bailey et al., Tetra. Lett. 2008, 49, 2159; and Fensome et al., Bioorg. Med. Chem. Lett. 2010, 20, 1555) to afford a compound of Formula I of this invention.

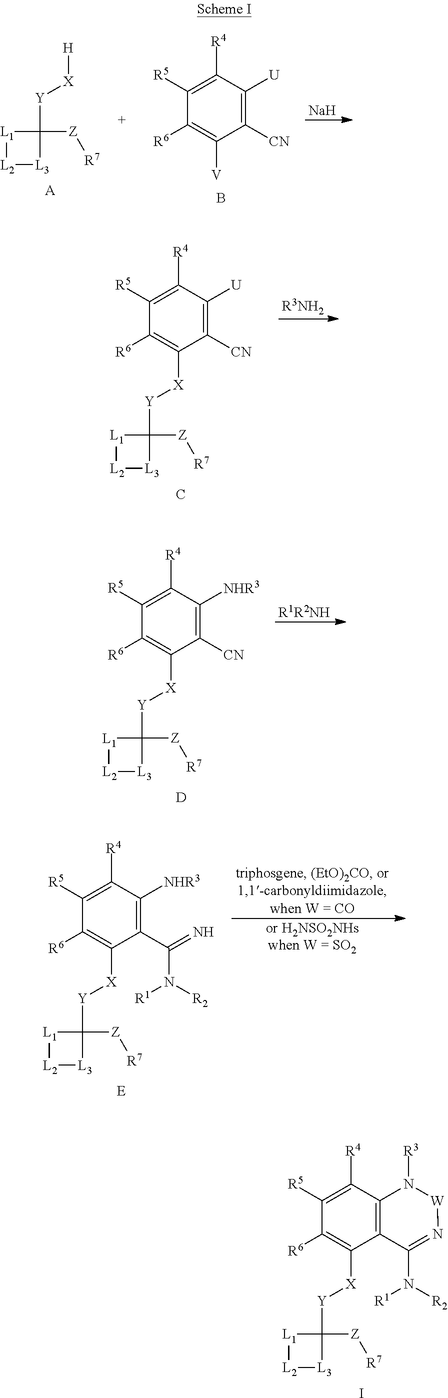

In Scheme I, each of L¹, L², L³, R¹, R², R³, R⁴, R⁵, R⁶, R⁷, W, X, Y, and Z is as defined above. U and V, independently, are a leaving group such as halo (e.g., F, Cl, Br, and I; preferably F) and OSO₂R^{f} (R^{f} being perfluoroalkyl or aryl, e.g., OSO₂CF₃).

Each step shown in Scheme I adds a moiety toward the final product of Formula I. The order of the steps can be altered so that the moieties are added in an order to forming the final product.

As an illustration, a compound of Formula D can be prepared by reacting a compound of Formula A with a compound of Formula C-I:

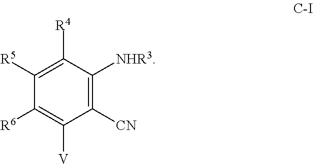

In this compound, V is halo or OSO₂R^{f}.

A compound of Formula E can be prepared using one of the following routes:

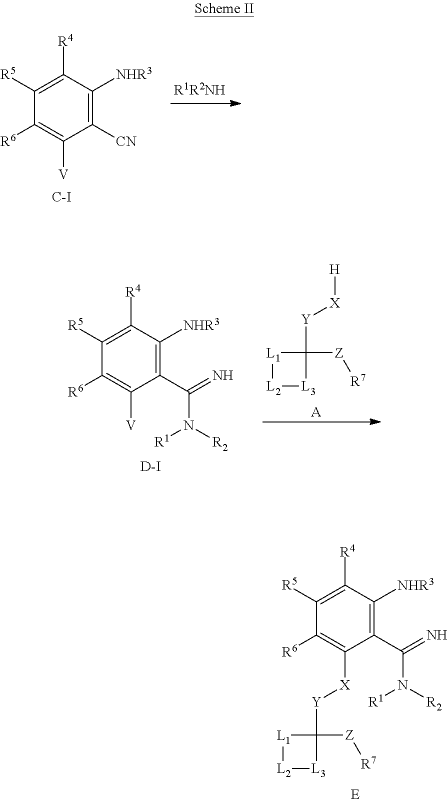

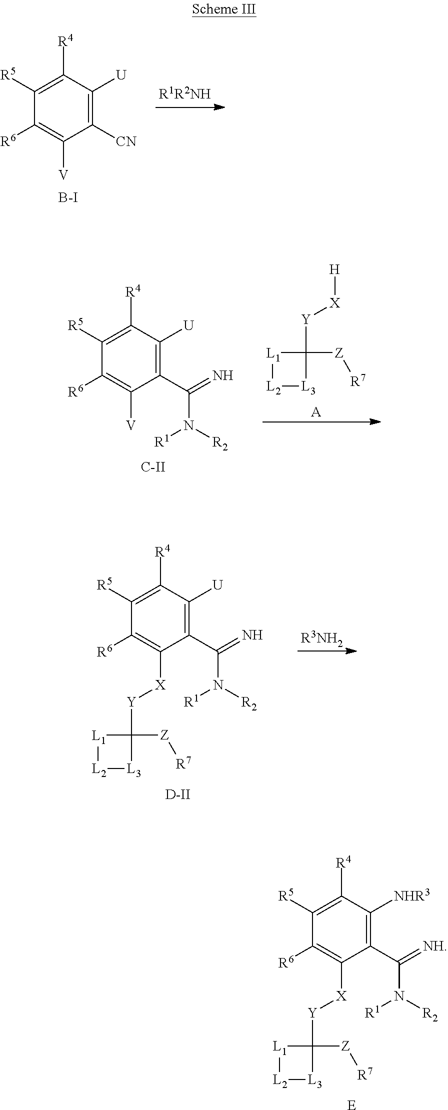

A compound of Formula I can be prepared via one of the following routes:

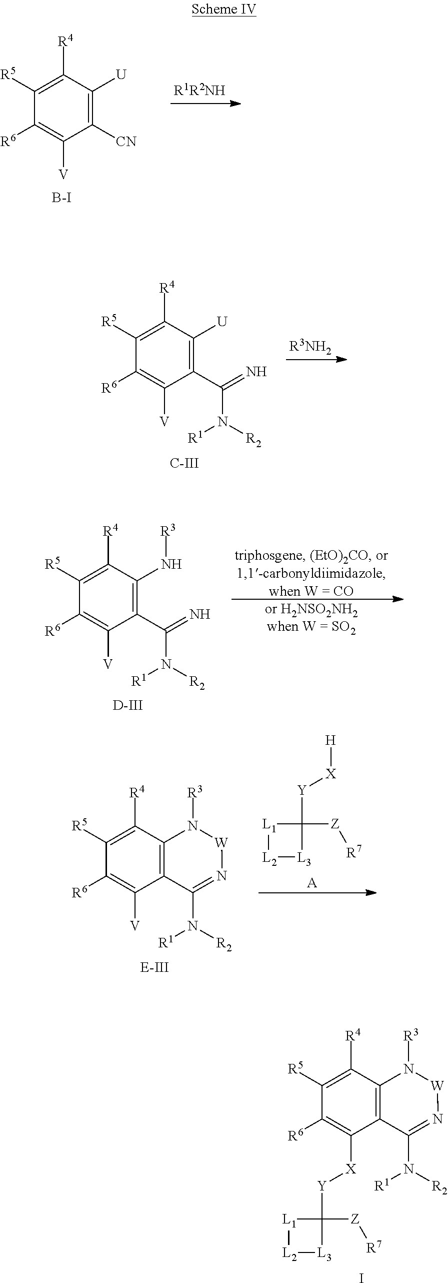

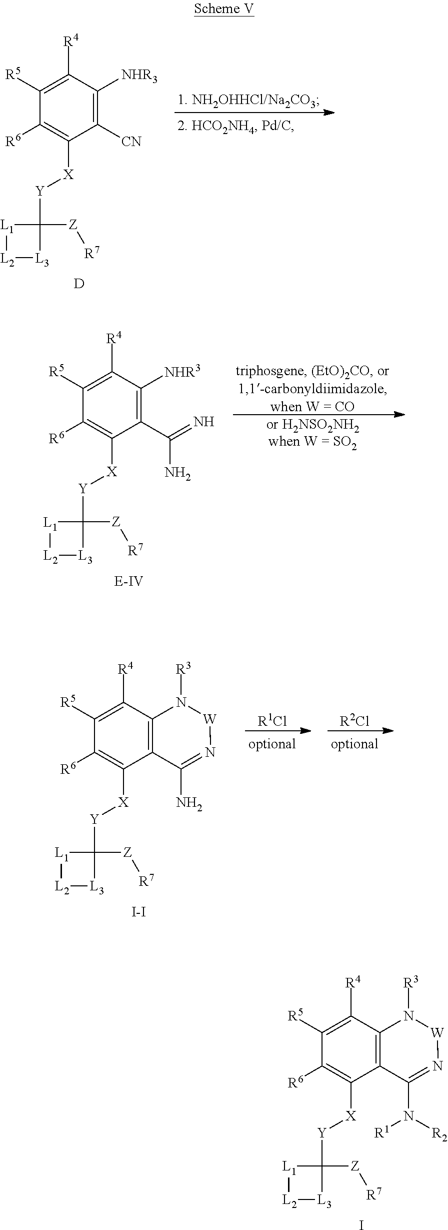

Accordingly, within the scope of this invention is a method of preparing a compound of Formula I comprising cyclizing a compound of Formula E or E-IV.

In some embodiments, when W in Formula I is C(O), the cyclizing is achieved by reacting the compound of Formula E with phosgene (COCl₂), triphosgene (CCl₃OCOOCCl₃), an organic carbonate (R^{b1}OCOR^{b2}), or 1,1′-carbonyldiimidazole. In other embodiments, when W is S(O)₂, cyclizing is achieved by reacting the compound of Formula E with sulfuric diamide (NH₂SO₂NH₂), sulfuryl chloride (SO₂Cl₂), 1,1′-sulfonyldiimidazole, or an organosulfate

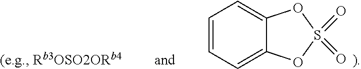 [SMILES: O=S1(=O)Oc2ccccc2O1]

Each of R^{b1}, R^{b2}, R^{b3}, and R^{b4}, independently, is C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ acyl, aryl, C₁-C₁₀ arylalkyl, heteroaryl, C₁-C₁₀ heteroarylalkyl, C₁-C₁₀ alkylsulfonyl, arylsulfonyl, or heteroarylsulfonyl. Examples include methyl, ethyl, and phenyl.

In other embodiments, when W is S(O)₂, the cyclizing is achieved by reacting the compound of Formula E with sulfuric diamide, sulfuryl chloride, 1,1′-sulfonyldiimidazole, or an organosulfate.

Also within the scope of this invention Compounds of Formulas E and E-IV. In one subset of these compounds, L¹ is C(O)NH, and L¹, L², L³, and the atom to which L¹ and L³ attached form a 5 or 6-membered heterocycloalkyl ring. In another subset of the compounds, X is O and Y is CH₂. Preferred compounds are those in which each of R¹, R², R³, R⁴, R⁵, and R⁶ is H; R⁷ is C₃-C₁₀ cycloalkyl, C₁-C₁₀ heterocycloalkyl, C₁-C₁₀ alkyl, or C₁-C₁₀ heteroalkyl; and Z is C(O)NH.

Still within the scope of this invention are compounds of Formula D and a method of preparing a compound of Formula E by reacting R¹R²NH with a compound of Formula D.

The compounds of this invention can also be prepared using other suitable starting materials through the synthetic route above and others known in the art. The method set forth above can include one or more additional steps to add or remove suitable protecting groups. In addition, various synthetic steps can be performed in an alternate order to obtain the desired compounds. Synthetic chemistry transformations and protecting group methodologies (protection and deprotection) useful in synthesizing the starting materials and intermediates are known in the art, including, for example, R. Larock, Comprehensive Organic Transformations (2^{nd} Ed., VCH Publishers 1999); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2^{nd} ed., John Wiley and Sons 2009) and subsequent editions thereof.

Certain compounds of this invention may contain a non-aromatic double bond and one or more asymmetric centers. Thus, the chemical structures depicted herein encompass all possible stereoisomers (i.e., enantiomers, diastereomers, and cis- or trans-isomers) and stereoisomeric mixtures. Stereoisomeric mixtures can be resolved into their component enantiomers, diastereomers, or cis/trans-isomers using separation techniques or chiral synthesis techniques well known to the skilled artisan.

The compounds of this invention include the compounds themselves as well as tautomers, salts, solvates, amides, and/or esters thereof.

The term “tautomer” as used herein refers to constitutional isomers that can readily change into one another by migration of an atom or group (e.g., H) so that they can exist together in equilibrium. The compounds may also exist in several tautomeric forms including the enol form, the keto form, and mixtures thereof. Accordingly, the chemical structures depicted herein encompass all possible tautomeric forms of the illustrated compounds.

“Salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

Compounds described herein include their unsolvated forms as well as solvated forms, including hydrated forms and as N-oxides. In general, compounds may be hydrated, solvated, or N-oxides. The compounds may also exist in multiple crystalline or amorphous forms. In general, all physical forms are equivalent for the uses contemplated herein and are intended to be within the scope of the present invention.

“Solvate” means a compound formed by solvation (the combination of solvent molecules with molecules or ions of the solute), or an aggregate that is composed of a solute ion or molecule, i.e., a compound of the present invention, with one or more solvent molecules. When water is the solvent, the corresponding solvate is “hydrate”.

“N-oxide,” also known as amine oxide or amine-N-oxide, means a compound that derives from a compound of the present invention via oxidation of an amine group of the compound of the present invention. An N-oxide typically contains the functional group R₃N⁺—O⁻ (sometimes written as R₃N═O or R₃N→O).

Not to be bound by any theory, the compounds of the present invention are chemosensory receptor ligand modulators in the sense that the compounds modulate or increase the binding between a sweetener (e.g., glucose) and a chemosensory receptor (e.g., a sweet receptor). Herein such a compound will enhance the activation of a sweet receptor by its ligand, i.e., glucose. More specifically, the compounds of the invention are capable of interacting with both a sweetener (e.g., glucose) and a sweet receptor or modulating the structure or function of the receptor, to activate or increase the signal transduction activity of the receptor. The term “capable of interacting with” or “interacting with” used herein means that a compound binds to or forms one or more molecular interactions, e.g., productive interactions with another molecule such as a sweet receptor. Exemplary molecular interactions include van der Waals, burial of hydrophobic atoms or atomic groups, hydrogen bonds, ring stacking interactions, salt-bridging electrostatic interactions, or a combination thereof. The glucose enhancing effect of these compounds can be judged by animal or human studies such as a panel of human taste testers, or via procedures commonly known in the field.

In one embodiment, the present invention provides a consumable composition, which includes a compound of the present invention. In another embodiment, the compound is Compound 1, 2, or 3, or a tautomer, salt, solvate, and/or ester thereof. In certain embodiments, the consumable composition contains glucose. In other embodiments, the consumable composition further includes one or more sweeteners in addition to glucose.

Further, the present invention provides a method of enhancing the sweet taste of a consumable composition by contacting the consumable composition or a precursor thereof with a compound of the invention to form a modified consumable composition. In some embodiments, the consumable composition contains glucose. In particular embodiments, the compound in the consumable composition is compound 1, 2, or 3, or a tautomer, salt, solvate, and/or ester thereof.

In some embodiments, the compounds of the present invention can be used at a level of 100 parts per billion to 1000 parts per million, in combination with glucose so as to reduce the concentration of glucose required to prepare a consumable composition having the desired degree of sweetness.

In addition to glucose, the compound of the present invention can also be used in combination with other known natural or artificial sweeteners including, but not limited to, the common saccharide sweeteners, e.g., sucrose, fructose, and sweetener compositions comprising natural sugars, such as corn syrup (including high fructose corn syrup) or other syrups or sweetener concentrates derived from natural fruit and vegetable sources, semi-synthetic “sugar alcohol” sweeteners such as erythritol, isomalt, lactitol, mannitol, sorbitol, xylitol, maltodextrin, and the like, and artificial sweeteners such as aspartame, saccharin, acesulfame-K, cyclamate, sucralose, and alitame. Sweeteners also include cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A and other sweet Stevia-based glycosides, carrelame and other guanidine-based sweeteners, etc. The term “sweeteners” also includes combinations of sweeteners just described above.

As used herein, a “consumable composition” includes any substance intended for oral consumption either alone or together with another substance. The consumable composition includes both “food or beverage products” and “non-edible products”. By “Food or beverage products”, it is meant any edible product intended for consumption by humans or animals, including solids, gel, paste, foamy material, semi-solids, liquids (e.g., beverages), or mixtures thereof. The term “non-edible products” or “nonconsumable composition” includes supplements, nutraceuticals, functional food products (e.g., any fresh or processed food claimed to have a health-promoting and/or disease-preventing properties beyond the basic nutritional function of supplying nutrients), pharmaceutical and over the counter medications intended to provide a therapeutic benefit, oral care products such as dentifrices and mouthwashes, cosmetic products such as sweetened lip balms and other personal care products that use glucose and or other sweeteners. In this respect, a “nonconsumable composition” is intended to be consumed or used by humans or animals; however, it is not consumed as food. A nonconsumable composition includes solids, gel, paste, foamy material, semi-solids, liquids, or mixtures thereof. By “animal”, it includes any non-human animal, such as, for example, farm animals and pets.

A consumable composition also includes over-the-counter (OTC) product, i.e., a product for household and/or personal use which may be sold without a prescription and/or without a visit to a medical professional. Examples of the OTC products include, but are not limited to, vitamins and dietary supplements; topical analgesics and/or anesthetic; cough, cold and allergy remedies; antihistamines and/or allergy remedies; and combinations thereof. Vitamins and dietary supplements include, but are not limited to, vitamins, dietary supplements, tonics/bottled nutritive drinks, child-specific vitamins, dietary supplements, any other products of or relating to or providing nutrition, and combinations thereof. Topical analgesics and/or anesthetics include any topical cream/ointment/gel used to alleviate superficial or deep-seated aches and pains, e.g. muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. Examples of oral hygiene product include, but are not limited to mouth cleaning strips, toothpaste, toothbrushes, mouthwashes/dental rinses, denture care, mouth fresheners, at-home teeth whiteners, and dental floss.

As indicated, the compounds of the invention can be added to food or beverage products or formulations. Examples of food and beverage products or formulations include, but are not limited to, sweet coatings, frostings, or glazes for consumable products or any entity included the in-the-soup category, the dried processed food category, the beverage category, the ready meal category, the canned or preserved food category, the frozen processed food category, the chilled processed food category, the snack food category, the baked goods category, the confectionery category, the dairy product category, the ice cream category, the meal replacement category, the pasta and noodle category, and the sauces, dressings, condiments category, the baby food category, and/or the Spreads category.

In general, the soup category refers to canned/preserved, dehydrated, instant, chilled, UHT and frozen soup. For the purpose of this definition, the term “soup” means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomme) to sauces (cream or cheese-based soups).

Dehydrated and culinary food category usually means cooking aid products, meal solutions products, or meal embellishment products. Cooking aid products include, e.g., powders, granules, pastes, concentrated liquid products (e.g., concentrated bouillon), bouillon and bouillon like products in pressed cubes, tablets or powder or granulated form, which are sold separately as a finished product or as an ingredient within a product, sauces and recipe mixes (regardless of technology). Meal solutions products include, e.g., dehydrated and freeze dried soups, including dehydrated soup mixes, dehydrated instant soups, dehydrated ready-to-cook soups, dehydrated or ambient preparations of ready-made dishes, and meals and single serve entrees including pasta, potato, and rice dishes. Meal embellishment products include, e.g., condiments, marinades, salad dressings, salad toppings, dips, breading, batter mixes, shelf stable spreads, sauces or sauce mixes (e.g., barbecue sauces), liquid recipe mixes, concentrates, and recipe mixes for salad. Each of the products mentioned above can be sold as a finished product or as an ingredient within a product, whether dehydrated, liquid or frozen.

The beverage category usually means beverages, beverage mixes and concentrates, including, but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes alcoholic drinks, soft drinks, sports drinks, isotonic beverages, and hot drinks. Alcoholic drinks include, but are not limited to, beer, cider/perry, FABs, wine, and spirits. Soft drinks include, but are not limited to, carbonates, such as colas and non-cola carbonates; fruit juice, such as juice, nectars, juice drinks, and fruit flavored drinks; bottled water, which includes sparkling water, spring water, and purified/table water; functional drinks, which can be carbonated or still and include sport, energy, and elixir drinks; concentrates, such as liquid and powder concentrates in ready to drink measure. Hot drinks include, but are not limited to, coffee, such as fresh, instant, and combined coffee; tea, such as black, green, white, oolong, and flavored tea; and other hot drinks including flavor-, malt- or plant-based powders, granules, blocks, and tablets mixed with milk or water.

The sack food category generally refers to any food that can be a light informal meal including, but not limited to, sweet and savory snacks and snack bars. Examples of snack foods include, but are not limited to, fruit snacks, chips/crisps, extruded snacks, tortilla/corn chips, popcorn, pretzels, nuts, and other sweet and savory snacks. Examples of snack bars include, but are not limited to, granola/muesli bars, breakfast bars, energy bars, fruit bars, and other snack bars.

The baked goods category generally refers to any edible product, the process of preparing which involves exposure to heat or excessive sunlight. Examples of baked goods include, but are not limited to, bread, buns, cookies, muffins, cereal, toaster pastries, pastries, waffles, tortillas, biscuits, pies, bagels, tarts, quiches, cake, any baked foods, and any combination thereof.

The ice cream category generally refers to frozen dessert containing cream and sugar and flavoring. Examples of ice cream include, but are not limited to, impulse ice cream; take-home ice cream; frozen yogurt and artisanal ice cream; soy, oat, bean (e.g., red bean and mung bean), and rice-based ice creams.

The confectionery category generally refers to an edible product that is sweet to the taste. Examples of confectionery include, but are not limited to, candies, gelatins, chocolate confectionery, sugar confectionery, gum, and any combination thereof.

The meal replacement category generally refers to any food intended to replace normal meals, particularly for people having health or fitness concerns. Examples include, but are not limited to, slimming products and convalescence products.

The ready meal category generally refers to any food that can be served as a meal without extensive preparation or processing. Ready meals include products that have recipe “skills” added to them by the manufacturer, resulting in a high degree of readiness, completion and convenience. Examples of ready meals include, but are not limited to, canned/preserved, frozen, dried, chilled ready meals; dinner mixes; frozen pizza; chilled pizza; and prepared salads.

The pasta and noodle category includes any pastas and/or noodles including, but not limited to, canned, dried and chilled/fresh pasta; and plain, instant, chilled, frozen, and snack noodles.

The canned/preserved food category includes, but is not limited to, canned/preserved meat and meat products, fish/seafood, vegetables, tomatoes, beans, fruit, ready meals, soup, pasta, and other canned/preserved foods.

The frozen processed food category includes, but is not limited to, frozen processed red meat, processed poultry, processed fish/seafood, processed vegetables, meat substitutes, processed potatoes, bakery products, desserts, ready meals, pizza, soup, noodles, and other frozen food.

The dried processed food category includes, but is not limited to, rice, dessert mixes, dried ready meals, dehydrated soup, instant soup, dried pasta, plain noodles, and instant noodles.

The chill processed food category includes, but is not limited to, chilled processed meats, processed fish/seafood products, lunch kits, fresh cut fruits, ready meals, pizza, prepared salads, soup, fresh pasta, and noodles.

The sauces, dressings and condiments category includes, but is not limited to, tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The baby food category includes, but is not limited to, milk- or soybean-based formula; and prepared, dried and other baby food.

The spreads category includes, but is not limited to, jams and preserves, honey, chocolate spreads, nut-based spreads, and yeast-based spreads.

The dairy product category generally refers to edible product produced from mammal's milk. Examples of dairy product include, but are not limited to, drinking milk products, cheese, yoghurt and sour milk drinks, and other dairy products.

Additional examples for consumable compositions, particularly food and beverage products or formulations, are provided as follows: one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, licorice, other sugar confectionery, gum, chewing gum, sugarized gum, sugar-free gum, functional gum, bubble gum, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, ready-to-eat cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yogurt, plain/natural yogurt, flavored yogurt, fruited yogurt, probiotic yogurt, drinking yogurt, regular drinking yogurt, probiotic drinking yogurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary consumable compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, spreads, and a mixture thereof. Exemplary additional consumable compositions also include breakfast cereals, sweet beverages or solid, and liquid concentrate compositions for preparing beverages, ideally so as to enable the reduction in concentration of glucose and/or artificial sweeteners.

Typically at least a sweet receptor modulating amount, a sweet flavor modulating amount, or a sweet flavor enhancing amount of a compound of the present invention will be added to the consumable or medicinal product, optionally in the presence of glucose so that the sweet flavor modified consumable or medicinal product has an increased sweet taste as compared to the consumable or medicinal product prepared without the compound of the present invention, as judged by human beings or animals in general, or in the case of formulations testing, as judged by a majority of a panel of human taste testers, via procedures commonly known in the field.

A skilled person in the art can determine through in situ and/or in vivo assays the concentration of a compound of the invention needed to modulate or improve the flavor of the consumable or medicinal product or composition, considering variables including the specific type of consumable composition and its various other ingredients, especially the presence of glucose or other known sweet flavoring agents and the concentrations thereof, the natural genetic variability and individual preferences and health conditions of various human beings tasting the compositions, and the subjective effect of the particular compound on the taste of such chemosensory compounds.

One application of the compounds of the invention is for inducing or enhancing the sweet taste or other taste properties of glucose, and consumable compositions made therefrom. A broad but also low range of concentrations of the compounds of the present invention would typically be required, from 0.001 ppm to 100 ppm (e.g., 0.1 ppm to 10 ppm, 0.01 ppm to 30 ppm, 0.05 ppm to 10 ppm, 0.01 ppm to 5 ppm, 0.02 ppm to 2 ppm, and 0.01 ppm to 1 ppm).

In another application, the compounds of the invention can be provided in pharmaceutical compositions containing a therapeutically effective amount of one or more therapeutic agents together with a suitable amount of a pharmaceutically acceptable vehicle, so as to provide the form for proper administration to a patient. When administered to a patient, preferably the composition is sterile. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions including a compound of the present invention may be manufactured by means of conventional mixing, dissolving, granulating, dragee-making, levigating, emulsifying, encapsulating, entrapping, or lyophilizing processes. Pharmaceutical compositions may be formulated in conventional manner using one or more physiologically acceptable carriers, diluents, excipients or auxiliaries, which facilitate processing of compounds of the present invention into preparations which can be used pharmaceutically. Proper formulation depends upon the route of administration chosen.

Pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule (see e.g., U.S. Pat. No. 5,698,155). Other examples of suitable pharmaceutical vehicles have been described in the art (see Remington: The Science and Practice of Pharmacy, Philadelphia College of Pharmacy and Science, 20^{th} Edition, 2000).

When a compound of this invention is used in a consumable composition, the compound can be combined with conventional flavoring materials or adjuvants. Such conventional flavoring materials include saturated fatty acids, unsaturated fatty acids and amino acids; alcohols including primary and secondary alcohols; esters; carbonyl compounds including ketones (other than the 4-ethyloctanal derivatives of our invention) and aldehydes; lactones; other cyclic organic materials including benzene derivatives, alicyclic compounds, heterocyclics such as furans, pyridines, pyrazines, and the like; sulfur-containing compounds including thiols, sulfides, disulfides, and the like; proteins; lipids; carbohydrates; so-called flavor potentiators such as monosodium glutamate, magnesium glutamate, calcium glutamate, guanylates, and inosinates; natural flavoring materials such as cocoa, vanilla and caramel; essential oils and extracts such as anise oil, clove oil, and the like; and artificial flavoring materials such as vanillin, ethyl vanillin and the like.

Specific preferred flavor adjuvants include, but are not limited to, the following: anise oil; ethyl-2-methyl butyrate; vanillin; cis-3-heptenol; cis-3-hexenol; trans-2-heptenal; butyl valerate; 2,3-diethyl pyrazine; methyl cyclo-pentenolone; benzaldehyde; valerian oil; 3,4-dimeth-oxyphenol; amyl acetate; amyl cinnamate; γ-butyryl lactone; furfural; trimethyl pyrazine; phenyl acetic acid; isovaleraldehyde; ethyl maltol; ethyl vanilin; ethyl valerate; ethyl butyrate; cocoa extract; coffee extract; peppermint oil; spearmint oil; clove oil; anethol; cardamom oil; wintergreen oil; cinnamic aldehyde; ethyl-2-methyl valerate; γ-hexenyl lactone; 2,4-decadienal; 2,4-heptadienal; methyl thiazole alcohol (4-methyl-5-β-hydroxyethyl thiazole); 2-methyl butanethiol; 4-mercapto-2-butanone; 3-mercapto-2-pentanone; 1-mercapto-2-propane; benzaldehyde; furfural; furfuryl alcohol; 2-mercapto propionic acid; alkyl pyrazine; methyl pyrazine; 2-ethyl-3-methyl pyrazine; tetramethyl pyrazine; polysulfides; dipropyl disulfide; methyl benzyl disulfide; alkyl thiophene; 2,3-dimethyl thiophene; 5-methyl furfural; acetyl furan; 2,4-decadienal; guiacol; phenyl acetaldehyde; β-decalactone; d-limonene; acetoin; amyl acetate; maltol; ethyl butyrate; levulinic acid; piperonal; ethyl acetate; n-octanal; n-pentanal; n-hexanal; diacetyl; monosodium gulatamate; monopotassium glutamate; sulfur-containing amino acids, e.g., cysteine; hydrolyzed vegetable protein; 2-methylfuran-3-thiol; 2-methyldihydrofuran-3-thiol; 2,5-dimethylfuran-3-thiol; hydrolyzed fish protein; tetramethyl pyrazine; propylpropenyl disulfide; propylpropenyl trisulfide; diallyl disulfide; diallyl trisulfide; dipropenyl disulfide; dipropenyl trisulfide; 4-methyl-2-[(methylthio)-ethyl]-1,3-dithiolane; 4,5-dimethyl-2-(methylthiomethyl)-1,3-dithiolne; and 4-methyl-2-(methylthiomethyl)-1,3-dithiolane. These and other flavor ingredients are described in U.S. Pat. Nos. 6,110,520 and 6,333,180, both of which are hereby incorporated by reference.

A compound of the invention or compositions incorporating the same can be combined with one or more vehicles or carriers for adding the compound to the particular product. Vehicles can be edible or otherwise suitable materials such as ethyl alcohol, propylene glycol, water, and the like, as described supra. Carriers include materials such as gum arabic, carrageenan, xanthan gum, guar gum, and the like.

The following is provided to show that Compounds 1-3 of this invention unexpectedly increase the sweet taste of glucose.

### Example 1

### Preparation of 3-(((4-Amino-2,2-dioxido-1H-benzo[c][1,2,6]thiadiazin-5-yl)oxy)methyl)-N-cyclopentyl-2-oxopiperidine-3-carboxamide (Compound 1)

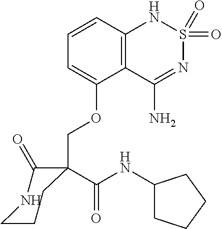 [SMILES: NC1=NS(=O)(=O)Nc2cccc(OCC3(C(=O)NC4CCCC4)CCCNC3=O)c21]

Compound 1 was prepared using the steps shown in Schemes I and V above.

More specifically, a mixture of 3-((3-amino-2-carbamimidoylphenoxy)-methyl)-N-cyclopentyl-2-oxopiperidine-3-carboxamide (65 mg, 0.174 mmol) and sulfuric diamide (167 mg, 1.74 mmol) in dioxane (10 mL) was heated at 180° C. under microwave for 60 minutes. The mixture was concentrated, filtered, and washed with water, EtOAc, and dried to give Compound 1 as a tan solid (47 mg, 62%). ¹H NMR (400 MHz, DMSO-d₆) δ 1.28-1.88 (m, 11H), 2.35-2.45 (m, 1H), 3.13-3.25 (m, 2H), 3.95-4.08 (m, 1H), 4.31-4.43 (m, 2H), 6.64 (d, J=8.16 Hz, 1H), 6.73 (d, J=7.92 Hz, 1H), 7.45 (dd, J=8.16, 7.92 Hz, 1H), 7.70 (d, J=7.15 Hz, 1H), 8.19 (s, 1H), 8.33 (s, 1H), 8.25 (s, 1H), 10. 94 (s, 1H). MS 436 (MH⁺).

### Example 1a

### Preparation of Intermediate 3-((3-Amino-2-carbamimidoylphenoxy)-methyl)-N-cyclopentyl-2-oxopiperidine-3-carboxamide

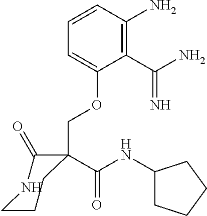 [SMILES: N=C(N)c1c(N)cccc1OCC1(C(=O)NC2CCCC2)CCCNC1=O]

This intermediate compound was prepared following the procedure below.

A suspension of 3-((3-amino-2-(N-hydroxycarbamimidoyl)phenoxy)methyl)-N-cyclopentyl-2-oxopiperidine-3-carboxamide (100 mg, 0.257 mmol; preparation described below) and zinc powder (168 mg, 2.57 mmol) in HOAc (10 mL) was vigorously stirred at 80° C. for 30 minutes. The mixture was diluted with EtOAc, washed with water, brine. The organic layer was separated and dried over MgSO₄, filtered, concentrated to provide 3-((3-amino-2-carbamimidoylphenoxy)methyl)-N-cyclopentyl-2-oxopiperidine-3-carboxamide (85 mg, 89%) as a brown solid. ¹H NMR (400 MHz, DMSO-d₆) δ 1.27-2.00 (m, 11H), 2.29-2.41 (m, 1H), 3.15-3.19 (m, 2H), 3.87 (d, J=8.6 Hz, 1H), 3.91-4.16 (m, 1H), 4.38 (d, J=8.6 Hz, 1H), 5.37 (bs, 2H), 6.29 (d, J=8.2 Hz, 1H), 6.39 (d, J=8.2 Hz, 1H), 7.10 (dd, J=8.2, 8.2 Hz, 1H), 7.62 (d, J=7.2 Hz, 1H), 8.00 (bs, 1H), 9.28-8.60 (m, 3H). MS 374 (MH⁺).

### Example 1b

### 3-((3-Amino-2-(N-hydroxycarbamimidoyl)phenoxy)methyl)-N-cyclopentyl-2-oxopiperidine-3-carboxamide

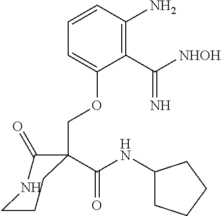

A mixture 3-((3-amino-2-cyanophenoxy)methyl)-N-cyclopentyl-2-oxopiperidine-3-carboxamide (1 g, 2.81 mmol; preparation described below) and hydroxylamine (25% solution in water; 140 mmol) in EtOH (20 mL) was refluxed overnight. After it was cooled to room temperature, the reaction mixture was concentrated under reduced pressure. The residue was extracted with EtOAc (3 times). The combined organic layers were dried over MgSO₄, concentrated, and purified by chromatography on silica gel eluting with DCM/MeOH from 1/0 to 92/8 to afford 3-((3-amino-2-(N-hydroxycarbamimidoyl)phenoxy)methyl)-N-cyclopentyl-2-oxopiperidine-3-carboxamide (0.76 g, 69%) as a white solid. ¹H NMR (500 MHz, DMSO-d₆) δ 1.26-1.66 (m, 7H), 1.73-1.93 (m, 4H), 2.30-2.38 (m, 1H), 3.11-3.19 (m, 2H), 3.97 (d, J=8.8 Hz, 1H), 3.94-4.02 (m, 1H), 4.24 (d, J=8.8 Hz, 1H), 5.42 (s, 4H), 6.20 (d, J=7.9 Hz, 1H), 6.31 (d, J=8.2 Hz, 1H), 6.94 (dd, J=8.2, 7.9 Hz, 1H), 7.58 (d, J=7.3 Hz, 1H), 7.97 (bs, 1H), 9.33 (s, 1H). MS 390 (MH⁺).

### Example 1c

### 3-((3-Amino-2-cyanophenoxy)methyl)-N-cyclopentyl-2-oxopiperidine-3-carboxamide

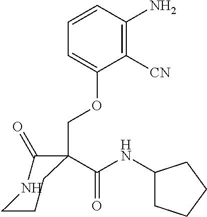 [SMILES: N#Cc1c(N)cccc1OCC1(C(=O)NC2CCCC2)CCCNC1=O]

A mixture of 3-((2-cyano-3-fluorophenoxy)methyl)-N-cyclopentyl-2-oxopiperidine-3-carboxamide (50 g, 139 mmol; preparation described below) and ammonium hydroxide (395 g, 4174 mmol) in isopropyl alcohol (160 mL) was heated at 120° C. in an autoclave overnight. After it was cooled down to room temperature, the precipitate was collected by filtration, washed with ether, and dried to give the title compound as a tan solid (35.0 g, 70%). ¹H NMR (400 MHz, DMSO-d₆) δ 1.29-2.03 (m, 11H), 2.35-2.43 (1H), 3.15-3.23 (m, 2H), 3.94 (d, J=8.77 Hz, 1H)), 3.95-4.04 (m, 1H), 4.45 (d, J=8.77 Hz, 1H), 5.99 (s, 2H), 6.22 (d, J=8.13 Hz, 1H), 6.34 (d, J=8.36 Hz, 1H), 7.17 (dd, J=8.13, 8.36 Hz, 1H), 7.58 (d, J=7.20 Hz, 1H), 8.05 (s, 1H). MS 357 (MH⁺).

### Example 1d

### 3-((2-Cyano-3-fluorophenoxy)methyl)-N-cyclopentyl-2-oxopiperidine-3-carboxamide

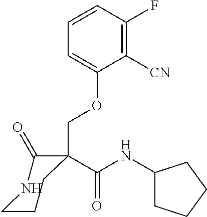 [SMILES: N#Cc1c(F)cccc1OCC1(C(=O)NC2CCCC2)CCCNC1=O]

To a solution of N-cyclopentyl-3-(hydroxymethyl)-2-oxopiperidine-3-carboxamide (62 g, 258 mmol; preparation described below) in dry THF (1000 mL) was added NaH (60% in mineral oil, 13.4 g, 335 mmol) in small portions at 0 OC under nitrogen. After it was stirred at room temperature for another 1 hr, the reaction mixture was cooled down to 0 OC in an ice-water bath. And to this solution was added 2,6-difluorobenzonitrile (46.7 g, 335 mmol). The resulting mixture was stirred at room temperature under nitrogen overnight. The reaction was quenched with brine, and extracted with EtOAc (3×500 mL). The combined organic layers were dried over MgSO₄, filtered, and concentrated under reduced pressure. The resultant residue was triturated with ether, filtered, and dried to afford the title compound (87.3 g, 94%) as a pale yellow solid. ¹H NMR (500 MHz, DMSO-d₆) δ 1.32-1.87 (m, 10H), 1.92-2.02 (m, 1H), 2.40-2.48 (m, 1H), 3.15-3.25 (m, 2H), 3.97-4.05 (m, 1H), 4.19 (d, J=8.88 Hz, 1H), 4.64 (d, J=8.88 Hz, 1H), 7.08 (d, J=8.70 Hz, 1H), 7.17 (d, J=8.70 Hz, 1H), 7.62 (d, J=7.20 Hz, 1H), 7.66-7.78 (m, 1H), 8.13 (s, 1H). MS 360 (MH⁺).

### Example 1e

### N-Cyclopentyl-3-(hydroxymethyl)-2-oxopiperidine-3-carboxamide

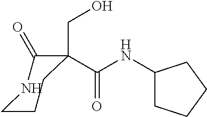 [SMILES: O=C1NCCCC1(CO)C(=O)NC1CCCC1]

To a suspension of N-cyclopentyl-2-oxopiperidine-3-carboxamide (164 g, 780 mmol; preparation described below) and paraformaldehyde (28.1 g, 936 mmol) in THF (500 mL) was added KOH (0.44 g, 7.8 mmol) at room temperature. The reaction mixture was then heated to 70 OC. Once the bath temperature reached to 70 OC, the heating source was removed. After it was cooled down to room temperature, the precipitate was collected by filtration, and dried under vacuum to afford the title compound (162 g, 86%) as a white solid. ¹H NMR (400 MHz, CD₃OD) δ 1.39-1.51 (m, 2H), 1.57-1.78 (m, 5H), 1.81-2.00 (m, 4H), 2.36-2.43 (m, 1H), 3.26-3.31 (m, 2H), 3.67 (d, J=10.65 Hz, 1H), 3.92 (d, 1H, J=10.65 Hz, 1H), 4.07-4.16 (m, 1H). MS 240 (MH⁺).

### Example 1f

### N-Cyclopentyl-2-oxopiperidine-3-carboxamide

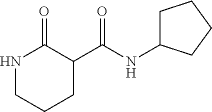 [SMILES: O=C1NCCCC1C(=O)NC1CCCC1]

A mixture of ethyl 2-oxopiperidine-3-carboxylate (150 g, 876 mmol; commercially available from TCI America) and cyclopentanamine (500 mL) was refluxed overnight. After it was cooled down to room temperature, the resultant precipitate was collected by filtration, washed with hexane, and dried under vacuum to give the title compound (164.0 g, 89%) as a white solid. ¹H NMR (500 MHz, CDCl₃) δ 1.65-1.48 (m, 2H), 1.52-2.00 (m, 9H), 2.32-2.40 (m, 1H), 3.14 (t, J=6.10 Hz, 1H), 3.26-3.36 (m, 2H), 4.13-4.22 (m, 1H), 7.28 (s, 1H), 7.62 (d, J=5.55 Hz, 1H). MS 211 (MH⁺).

### Example 2

### Preparation of 3-(((4-amino-2,2-dioxido-1H-benzo-[c][1,2,6]thiadiazin-5-yl)oxy)methyl)-N-isopropyl-2-oxopiperidine-3-carboxamide (i.e., Compound 2)

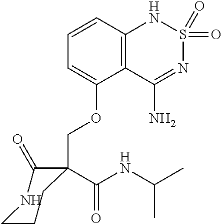 [SMILES: CC(C)NC(=O)C1(COc2cccc3c2C(N)=NS(=O)(=O)N3)CCCNC1=O]

Compound 2 was prepared following the same procedure as Compound 1 described above using 3-((3-amino-2-carbamimidoylphenoxy)methyl)-N-isopropyl-2-oxopiperidine-3-carboxamide (see below) and sulfuric diamide, with a yield of 81%. This compound is a white solid. ¹H NMR (400 MHz, DMSO-d₆) δ 1.02 (d, J=6.5 Hz, 3H), 1.05 (d, J=6.5 Hz, 3H), 1.47-1.64 (m, 1H), 1.67-1.86 (m, 2H), 2.34-2.46 (m, 1H), 3.10-3.24 (m, 2H), 3.79-3.93 (m, 1H), 4.18-4.44 (m, 2H), 6.62 (d, J=8.2 Hz, 1H), 6.71 (d, J=8.2 Hz, 1H), 7.44 (dd, J=8.2, 8.2 Hz, 1H), 7.57 (d, J=7.7 Hz, 1H), 8.16 (s, 1H), 8.24 (bs, 1H), 8.30 (bs, 1H), 10.91 (bs, 1H). MS 410 (MH⁺).

### Example 2a

### 3-((3-Amino-2-carbamimidoylphenoxy)methyl)-N-isopropyl-2-oxopiperidine-3-carboxamide

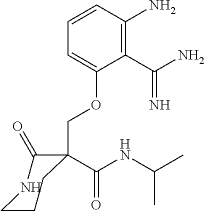 [SMILES: CC(C)NC(=O)C1(COc2cccc(N)c2C(=N)N)CCCNC1=O]

This compound was prepared following the procedure as in Example 1a from 3-((3-amino-2-(N-hydroxycarbamimidoyl)-phenoxy)-methyl)-N-isopropyl-2-oxopiperidine-3-carboxamide (Example 2b below) and zinc powder in acetic acid as a brown solid (81%). MS 348 (MH⁺).

### Example 2b

### 3-((3-amino-2-carbamimidoylphenoxy)methyl)-N-isopropyl-2-oxopiperidine-3-carboxamide

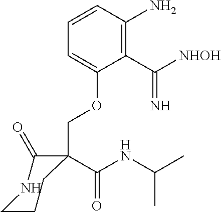

This compound was prepared following the procedure as in Example 1b from 3-((3-amino-2-cyanophenoxy)methyl)-N-isopropyl-2-oxopiperidine-3-carboxamide (Example 2c) and hydroxylamine in ethanol as a white solid (99%). MS 364 (MH⁺).

### Example 2c

### 3-((3-amino-2-cyanophenoxy)methyl)-N-isopropyl-2-oxopiperidine-3-carboxamide

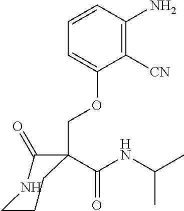 [SMILES: CC(C)NC(=O)C1(COc2cccc(N)c2C#N)CCCNC1=O]

This compound was prepared following the procedure as in Example 1c from 3-((2-cyano-3-fluorophenoxy)methyl)-N-isopropyl-2-oxopiperidine-3-carboxamide (Example 2d) and ammonia in isopropanol as a white solid (68%). ¹H NMR (400 MHz, DMSO-d₆) δ 1.05 (d, J=5.9 Hz, 3H), 1.07 (d, J=5.9 Hz, 3H), 1.47-1.71 (m, 1H), 1.72-1.88 (m, 1H), 1.88-2.03 (m, 1H), 2.30-2.44 (m, 1H), 3.03-3.24 (m, 2H), 3.77-3.90 (m, 1H), 3.94 (d, J=8.8 Hz, 1H), 4.46 (d, J=8.8 Hz, 1H), 5.90-6.09 (m, 2H), 6.22 (d, J=7.8 Hz, 1H), 6.35 (d, J=8.4 Hz, 1H), 7.10-7.22 (m, 1H), 7.47 (d, J=7.7 Hz, 1H), 8.02 (s, 1H). MS 331 (MH⁺).

### Example 2d

### 3-((2-cyano-3-fluorophenoxy)methyl)-N-isopropyl-2-oxopiperidine-3-carboxamide

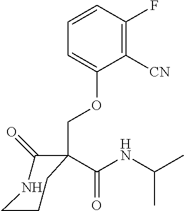 [SMILES: CC(C)NC(=O)C1(COc2cccc(F)c2C#N)CCCNC1=O]

This compound was prepared following the procedure as in Example 1d from 3-(hydroxymethyl)-N-isopropyl-2-oxopiperidine-3-carboxamide (Example 2e) and 2,6-difluorobenzonitrile as a white solid (91%). MS 334 (MH⁺).

### Example 2e

### 3-(hydroxymethyl)-N-isopropyl-2-oxopiperidine-3-carboxamide

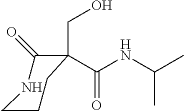

This compound was prepared following the procedure as in Example 1e from N-isopropyl-2-oxopiperidine-3-carboxamide (Example 2f) and paraformaldehyde as a white solid (77%). ¹H NMR (400 MHz, CD₃OD) δ 1.14 (d, J=6.4 Hz, 3H), 1.16 (d, J=6.4 Hz, 3H), 1.71 (m, 1H), 1.79-1.98 (m, 2H), 2.33-2.44 (m, 1H), 3.24-3.30 (m, 2H), 3.67 (d, J=10.7 Hz, 1H), 3.94 (d, J=10.7 Hz, 1H), 3.92-4.04 (m, 1H). MS 215 (MH⁺).

### Example 2f

### N-isopropyl-2-oxopiperidine-3-carboxamide

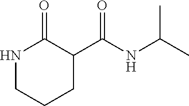

This compound was prepared following the procedure as in Example 1f from ethyl 2-oxopiperidine-3-carboxylate and isopropylamine as a white solid (100%). ¹H NMR (400 MHz, CDCl₃) δ 1.17 (d, J=6.5 Hz, 3H), 1.14 (d, J=6.5 Hz, 3H), 1.70-2.00 (m, 3H), 2.36 (m, 1H), 3.14 (t, J=6.0 Hz, 1H), 3.20-3.41 (m, 2H), 4.04 (m, 1H), 6.32 (bs, 1H), 7.48 (bs, 1H). MS 185 (MH⁺).

### Example 3

### Preparation of 3-(((4-amino-2,2-dioxido-1H-benzo[c][1,2,6]thiadiazin-5-yl)oxy)methyl)-N-cyclohexyl-2-oxopiperidine-3-carboxamide (i.e., Compound 3)

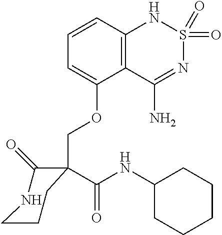 [SMILES: NC1=NS(=O)(=O)Nc2cccc(OCC3(C(=O)NC4CCCCC4)CCCNC3=O)c21]

Compound 3 was prepared following the procedure as in Example 1 from 3-((3-amino-2-carbamimidoylphenoxy)-methyl)-N-cyclohexyl-2-oxopiperidine-3-carboxamide (Example 3a) and sulfuric diamide as a white solid (80%). ¹H NMR (400 MHz, DMSO-d₆) δ 1.04-1.34 (m, 5H), 1.48-1.80 (m, 8H), 2.29-2.47 (m, 1H), 3.04-3.24 (m, 2H), 3.46-3.67 (m, 1H), 4.37 (q, J=9.3 Hz, 2H), 6.63 (d, J=8.3 Hz, 1H), 6.72 (d, J=8.3 Hz, 1H), 7.45 (t, J=8.3 Hz, 1H), 7.66 (d, J=7.7 Hz, 1H), 8.23 (s, 1H), 8.20 (s, 1H), 8.32 (bs, 1H), 10.92 (bs, 1H). MS 450 (MH⁺).

### Example 3a

### 3-((3-amino-2-carbamimidoylphenoxy)methyl)-N-cyclohexyl-2-oxopiperidine-3-carboxamide

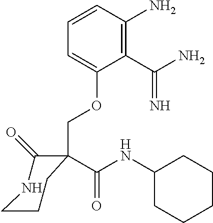 [SMILES: N=C(N)c1c(N)cccc1OCC1(C(=O)NC2CCCCC2)CCCNC1=O]

This compound was prepared following the procedure as in Example 1a from 3-((3-amino-2-(N-hydroxycarbamimidoyl)-phenoxy)-methyl)-N-cyclohexyl-2-oxopiperidine-3-carboxamide (Example 3b) and zinc powder in acetic acid as a pale-yellow solid (85%). MS 388 (MH⁺).

### Example 3b

### 3-((3-amino-2-(N-hydroxycarbamimidoyl)phenoxy)methyl)-N-cyclohexyl-2-oxopiperidine-3-carboxamide

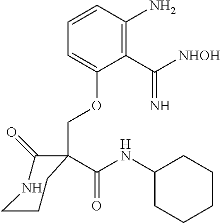

This compound was prepared following the procedure as in Example 1b from 3-((3-amino-2-cyanophenoxy)methyl)-N-cyclohexyl-2-oxopiperidine-3-carboxamide (Example 3c) and hydroxylamine in ethanol as a white solid (85%). MS 404 (MH⁺).

### Example 3c

### 3-((3-amino-2-cyanophenoxy)methyl)-N-cyclohexyl-2-oxopiperidine-3-carboxamide

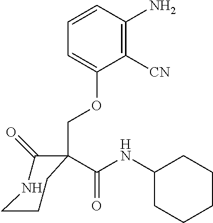 [SMILES: N#Cc1c(N)cccc1OCC1(C(=O)NC2CCCCC2)CCCNC1=O]

This compound was prepared following the procedure as in Example 1c from 3-((2-cyano-3-fluorophenoxy)methyl)-N-cyclohexyl-2-oxopiperidine-3-carboxamide (Example 3d) and ammonia in isopropanol as a white solid (66%). ¹H NMR (400 MHz, CD₃OD) δ 1.16-2.66 (m, 14H), 3.25-3.49 (m, 2H), 3.64-3.79 (m, 1H), 4.07 (d, J=8.5 Hz, 1H), 4.53 (d, J=8.5 Hz, 1H), 6.25 (d, J=8.3 Hz, 1H), 6.41 (d, J=8.3 Hz, 1H), 7.22 (t, J=8.3 Hz, 1H). MS 371 (MH⁺).

### Example 3d

### 3-((2-cyano-3-fluorophenoxy)methyl)-N-cyclohexyl-2-oxopiperidine-3-carboxamide

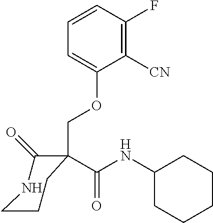 [SMILES: N#Cc1c(F)cccc1OCC1(C(=O)NC2CCCCC2)CCCNC1=O]

This compound was prepared following the procedure as in Example 1d from N-cyclohexyl-3-(hydroxymethyl)-2-oxopiperidine-3-carboxamide (Example 3e) and 2,6-difluorobenzonitrile as a white solid (91%). MS 374 (MH⁺).

### Example 3e

### N-cyclohexyl-3-(hydroxymethyl)-2-oxopiperidine-3-carboxamide

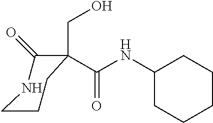 [SMILES: O=C1NCCCC1(CO)C(=O)NC1CCCCC1]

This compound was prepared following the procedure as in Example 1e from N-cyclohexyl-2-oxopiperidine-3-carboxamide (Example 3f) and paraformaldehyde as a white solid (98%). MS 255 (MH⁺).

### Example 3f

### N-cyclohexyl-2-oxopiperidine-3-carboxamide

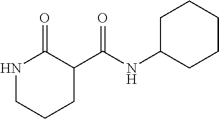 [SMILES: O=C1NCCCC1C(=O)NC1CCCCC1]

This compound was prepared following the procedure as in Example 1f from ethyl 2-oxopiperidine-3-carboxylate and cyclohexylamine as a white solid (97%). ¹H NMR (500 MHz, CDCl₃) δ 1.09-1.29 (m, 3H), 1.29-1.43 (m, 2H), 1.53-1.63 (m, 1H), 1.63-1.73 (m, 2H), 1.73-1.97 (m, 5H), 2.31-2.41 (m, 1H), 3.15 (t, J=6.1 Hz, 1H), 3.24-3.40 (m, 2H), 3.66-3.84 (m, 1H), 6.15 (bs, 1H), 7.43-7.67 (m, 1H). MS 225 (MH⁺).

### Example 4

### Glucose Enhancement Test

Compounds 1-3 were tested for glucose sweetness enhancement by human panelist taste tests. Test samples containing the compound of interest were tasted in pairs by the panelist to determine which of the samples were sweeter. Binominal distribution for two-sided pair comparisons were used to determine the probability of the correct number of responses at α=0.05 (95% confidence level).

The results indicated that (i) a composition containing 7% glucose+8 ppm Compound 1, 2, or 3 was perceived by panelists as being significantly sweeter than 8.5% glucose (p=0.00). See Tables 1 and 2 below. Further, the composition containing 7% glucose+8 ppm Compound 1 was perceived by panelists as sweet as 10% glucose (p>0.40), and (ii) the composition containing 7% glucose+8 ppm Compound 3 was perceived by panelists as sweet as 9% glucose (p>0.40).

TABLE 1

| Samples | Total | Significance |
|---|---|---|
| 7% Glucose + 8 ppm Compound 1 | 17 | 0.10 |
| 9.5% Glucose | 7 | |
| 7% Glucose + 8 ppm Compound 1 | 11 | >0.40 |
| 10% Glucose | 13 | |
| 7% Glucose + 8 ppm Compound 1 | 6 | 0.05 |
| 10.5% Glucose | 18 | |

TABLE 2
Sample selected as sweeter by panelists (12 panelists × 2 reps)

| Samples | Total | Significance |
|---|---|---|
| 7% Glucose + 8 ppm Compound 3 | 21 | 0.00 |
| 8.5% Glucose | 3 | |
| 7% Glucose + 8 ppm Compound 3 | 14 | >0.40 |
| 9% Glucose | 10 | |
| 7% Glucose + 8 ppm Compound 2 | 23 | 0.00 |
| 8.5% Glucose | 1 | |

By comparison, a close analog, the reported sucrose enhancer FEMA 4701 (CAS Number 1093200-92-0), only showed marginal glucose enhancement effects. Table 3 indicates that 7% glucose+8 ppm FEMA 4701 was perceived by panelists as being as sweet as 8% glucose (p>0.40), but significantly less sweet than 8.5% glucose (p<0.00).

Another reported sucrose enhancer FEMA 4774 (CAS Number 1359963-68-0) did not show significant glucose enhancement effects as indicated in Table 4 that 7% glucose+8 ppm FEMA 4774 was perceived by panelists as only being directionally sweeter than 7% glucose (p=0.10).

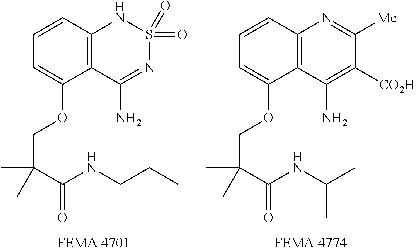

TABLE 3
Sample selected as sweeter by panelists (12 panelists × 2 reps)

| Samples | Total | Significance |
|---|---|---|
| 7% Glucose + 8 ppm FEMA 4701 | 13 | >0.40 |
| 8% Glucose | 11 | |
| 7% Glucose + 8 ppm FEMA 4701 | 21 | <0.00 |
| 8.5% Glucose | 3 | |
| 7% Glucose + 8 ppm FEMA 4774 | 17 | 0.10 |
| 7% Glucose | 7 | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Indeed, to achieve the purpose of enhancing the sweetness taste of any sweetener, one skilled in the art can design and prepare a compound that contains any combination of moieties described above and use it in a consumable product with any sweetener. Further, the ratio between the compound and the sweetener can be determined by a skilled artisan through assays known in the art so that the sweetness taste of the composition is enhanced.

From the above description, a skilled artisan can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A compound of Formula I: 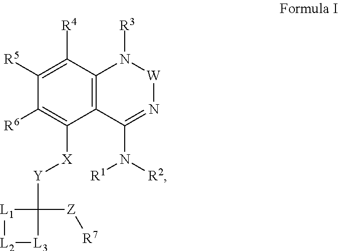
in which
W is S(O)₂ or C(O);
X is O, S, or NR^{a}, R^{a} being H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto;
Y is C₁-C₃ alkylene;
Z is CONR^{b}, R^{b} being H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto;
each of L¹, L², and L³, independently, is a bond, O, S, CO, C₁-C₆ alkylene, C₂-C₆ alkenylene, CONR^{c}, R^{d}NCONR^{e}, R^{f}NC(═NR^{g})NR^{h}, CRⁱR^{j}, or NR^{k}, provided that L¹, L², L³, and the carbon atom to which L¹ and L³ are attached form a 3- to 8-membered cycloalkyl or heterocycloalkyl ring, each of R^{c}, R^{d}, R^{e}, R^{f}, R^{g}, R^{h}, and R^{k}, independently, being H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto; and each of Rⁱ and R^{j}, independently, being H, OH, SH, CN, NO₂, NH₂, halo, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto;
each of R¹ and R², independently, is H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₃-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto; or R¹, R², and the N atom they attached to, together, form a 3 to 8-membered heterocycloalkyl or heteroaryl ring;
each of R³, and R⁷, independently, is H, OH, SH, NH₂, C₁-C₁₀ alkyl, C₂-C₁₀ alkenyl, C₂-C₁₀ alkynyl, C₁-C₁₀ heteroalkyl, C₃-C₈ cycloalkyl, C₃-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto; and
each of R⁴, R⁵, and R⁶, independently, is H, OH, SH, CN, NO₂, NH₂, halo, C₁-C₁₀ alkyl, C₂-C₁₀ alkenyl, C₂-C₁₀ alkynyl, C₁-C₁₀ heteroalkyl, C₃-C₈ cycloalkyl, C₁-C₈ heterocycloalkyl, C₁-C₁₀ alkoxy, C₁-C₁₀ alkylthio, C₁-C₁₀ acyl, C₁-C₁₀ acyloxy, aryl, aryloxy, arylthio, C₁-C₁₀ arylalkyl, heteroaryl, heteroaryloxy, heteroarylthio, C₁-C₁₀ heteroarylalkyl, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, C₁-C₁₀ alkylmercapto, or arylmercapto,
wherein
each of cycloalkyl, heterocycloalkyl, aryl, arylalkyl, heteroaryl, and heteroarylalkyl, independently, is unsubstituted or substituted with C₁-C₁₀ alkyl, C₂-C₁₀ alkenyl, C₂-C₁₀ alkynyl, C₃-C₈ cycloalkyl, C₁-C₈ heterocycloalkyl, C₁-C₁₀ alkoxy, C₁-C₁₀ alkylthio, aryl, aryloxy, arylthio, heteroaryl, heteroaryloxy, heteroarylthio, amino, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, hydroxyl, halogen, mercapto, C₁-C₁₀ alkylmercapto, arylmercapto, cyano, nitro, acyl, acyloxy, carboxyl, amido, carbamoyl, or carboxylic ester; and
each of alkyl, alkenyl, alkynyl, alkylene, and alkenylene is unsubstituted or substituted with C₃-C₈ cycloalkyl, C₁-C₈ heterocycloalkyl, C₁-C₁₀ alkoxy, C₁-C₁₀ alkylthio, aryl, aryloxy, arylthio, heteroaryl, heteroaryloxy, heteroarylthio, amino, C₁-C₁₀ alkylamino, C₁-C₂₀ dialkylamino, arylamino, diarylamino, heteroarylamino, diheteroarylamino, C₁-C₁₀ alkylsulfonyl, arylsulfonyl, heteroarylsulfonyl, C₁-C₁₀ alkylsulfonamide, arylsulfonamide, heteroarylsulfonamide, hydroxyl, halogen, mercapto, C₁-C₁₀ alkylmercapto, arylmercapto, cyano, nitro, acyl, acyloxy, carboxyl, amido, carbamoyl, or carboxylic ester.

2. The compound of claim 1, wherein each of R¹, R², R³, R⁴, R⁵, and R⁶ is H.

3. The compound of claim 1, wherein Y is CH₂.

4. The compound of claim 1, wherein W is SO₂.

5. The compound of claim 1, wherein X is O.

6. The compound of claim 1, wherein L¹, L², L³, and the atom to which L¹ and L³ attached form a 5 or 6-membered cycloalkyl or heterocycloalkyl ring.

7. The compound of claim 6, wherein X is O, Y is CH₂, and W is SO₂.

8. The compound of claim 1, wherein L¹ is C(O)NH, and L¹, L², L³, and the atom to which L¹ and L³ attached form a 5 or 6-membered heterocycloalkyl ring.

9. The compound of claim 8, wherein X is O, Y is CH₂, and W is SO₂.

10. The compound of claim 1, wherein Z is C(O)NH.

11. The compound of claim 1, wherein R⁷ is C₃-C₁₀ cycloalkyl, C₁-C₁₀ heterocycloalkyl, C₁-C₁₀ alkyl, or C₁-C₁₀ heteroalkyl.

12. The compound of claim 11, wherein each of R¹, R², R³, R⁴, R⁵, and R⁶ is H; R⁷ is cyclopentyl, cyclohexyl, or isopropyl; L¹ is C(O)NH; L¹, L², L³, and the atom to which L¹ and L³ attached form a 5 or 6-membered heterocycloalkyl ring; W is SO₂; X is O; Y is CH₂; and Z is C(O)NH.

13. The compound of claim 1, wherein the compound is Compound 1:

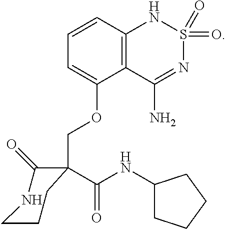 [SMILES: NC1=NS(=O)(=O)Nc2cccc(OCC3(C(=O)NC4CCCC4)CCCNC3=O)c21]

14. The compound of claim 1, wherein the compound is Compound 2:

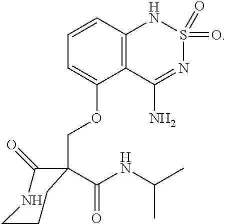 [SMILES: CC(C)NC(=O)C1(COc2cccc3c2C(N)=NS(=O)(=O)N3)CCCNC1=O]

15. The compound of claim 1, wherein the compound is Compound 3:

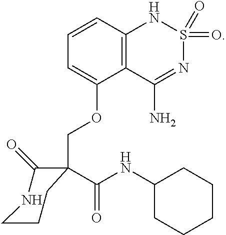 [SMILES: NC1=NS(=O)(=O)Nc2cccc(OCC3(C(=O)NC4CCCCC4)CCCNC3=O)c21]

16. A composition comprising a sweetener and a compound of claim 1, or a tautomer, salt, solvate, or ester thereof, wherein the compound is capable of enhancing the sweet taste of the sweetener.

17. The composition of claim 16, wherein the sweetener is glucose.

18. The composition of claim 16, wherein the compound is provided at a level of 1 part per billion or greater by weight.

19. The composition of claim 18, wherein the compound is provided at a level of 100 parts per billion to 100 parts per million by weight.

20. The composition of claim 16 further comprising a material selected from the group consisting of foodstuff, a chewing gum, a dental or oral hygiene product, and a medicinal product.

21. A method for enhancing the sweet taste of a consumable product, the method comprising mixing the consumable product with a compound of claim 1, wherein the consumable product contains a sweetener, and the compound of claim 1 is capable of enhancing the sweet taste of the sweetener.

22. The method of claim 21, wherein the compound of claim 1 is Compound 1, Compound 2, Compound 3, or a combination thereof.
